(19) United States
(12) Patent Application Publication
(10) Pub. No.: US 2018/0044685 A1
(43) Pub. Date: Feb. 15, 2018

(54) APPLICATION OF SYK SERVING AS THERAPEUTIC TARGET FOR HEPATIC FIBROSIS/HEPATIC CIRRHOSIS

(71) Applicant: Jian HONG, Guangzhou, Guangdong (CN)

(72) Inventor: Jian HONG, Guangzhou, Guangdong (CN)

(21) Appl. No.: 15/554,087

(22) PCT Filed: Aug. 11, 2016
(86) PCT No.: PCT/CN2016/094618

§ 371 Date: Aug. 28, 2017

(30) Foreign Application Priority Data
Sep. 24, 2015 (CN) 201510618201.2

(51) Int. Cl. C12N 15/65; A61K 38/00

(52) U.S. Cl. CPC C12N 15/65; A61K 38/005

(57) ABSTRACT

The present invention discloses an application of SYK serving as a therapeutic target for hepatic fibrosis/hepatic cirrhosis. Through experiments, the investor has found that a SYK gene has a higher expression during the process of hepatic fibrosis/hepatic cirrhosis, and the progression of hepatic fibrosis is accelerated by promoting activation of the hepatic stellate cells (HSCs); and the progression of hepatic fibrosis/hepatic cirrhosis can be effectively slowed down by using a SYK inhibitor or by interfering with SYK gene expression, both of which have good therapeutic effects.

### TECHNICAL FIELD

The present invention relates to a therapeutic target for hepatic fibrosis/hepatic cirrhosis.

### BACKGROUND

Hepatic fibrosis/hepatic cirrhosis represents a major health problem worldwide. The hepatic fibrosis is an essential stage for multiple chronic hepatic diseases developing into the hepatic cirrhosis, which refers to excessive or abnormal collagen fibers deposited in hepatic cell gaps resulted from imbalance between secretion and degradation of extracellular matrix when the liver is suffered from chronic injuries. The hepatic fibrosis refers to a pathological change at the early stage of the hepatic cirrhosis, during which the hepatic structure is changed and the supply of hepatic cells may be affected, thereby the hepatic function is gradually lost. If the hepatic fibrosis is not treated quickly, 25% to 40% thereof is finally developed into the hepatic cirrhosis or even a hepatic cancer, causing the hepatic failure, which may directly or indirectly cause death of patients.

The hepatic fibrosis is a compensatory repair response which has a chronic or progressive process. Most of chronic hepatic diseases caused by various etiologies are accompanied with the hepatic fibrosis. In western countries, alcoholic live injuries are a major cause for the hepatic fibrosis; while in Southeast Asia and our country, the hepatic fibrosis is mainly caused by persistent infection of hepatitis viruses, and in particular HBV. In China, 70% to 90% of hepatic cancers related to HBV are accompanied with the hepatic fibrosis/hepatic cirrhosis and some go through a process of “hepatitis-hepatic fibrosis/hepatic cirrhosis-hepatic cancer”.

Once formed, a hepatic cirrhosis state fails to reverse; however, a hepatic fibrosis stage must be undergone in its pathology forming process, and the reversibility of the hepatic fibrosis has been verified and recognized by many scholars at home and abroad. A formation mechanism of the hepatic fibrosis is relatively complicated, wherein hepatic stellate cells (HSCs) are crucial to generate and develop the hepatic fibrosis, and HSCs activation is a key link of generating the hepatic fibrosis. The HSCs are major matrix cells in a hepatic microenvironment, accounting for about 10% of hepatic innate cells. Down-regulating the activation of the HSCs or promoting apoptosis thereof becomes an important research orientation in exploring an effective treatment of the hepatic fibrosis on the premise of removing injury factors (such as an anti-hepatitis-virus therapy, alcohol withdrawal and the like).

At present, there are no effective therapeutic measures to treat the hepatic fibrosis yet; and a classic hepatic protection and an anti-hepatic fibrosis therapy also fail to bring about expected therapeutic effects clinically. Clinic and experimental studies on the hepatic fibrosis reserving mechanism have been continuously emerged in recent years, but a practical and effective therapeutic method has not been found yet. Therefore, to find a therapy means for reversing the process of hepatic fibrosis has a great clinical significance on effectively preventing generating the hepatic fibrosis or even developing the hepatic cancer, as well as improving patient's quality of life.

A tyrosine kinase is a kind of kinases widely existed in vertebrates, which can be classified into three types including a receptor tyrosine kinase, a cytoplast tyrosine kinase and an intranuclear tyrosine kinase. There is a vast number of tyrosine kinases, wherein more than fifty kinds of receptor tyrosine kinases have been found in vertebrates, while the cytoplast tyrosine kinases and the intranuclear tyrosine kinases include Src family, Tec family, ZAP70 family, JAK family, Abl family and other multi-gene superfamilies. Tyrosine kinase-mediated protein-tyrosine residue phosphorylation is an important process of regulating and controlling cell signaling pathways. Generation and development of various diseases of human are related to the expression and activity abnormality of tyrosine kinase, so that to develop targeted drugs in response to it has become a hot orientation of medical research in recent years. A large number of tyrosine kinase inhibitors have been applied to clinical practice, such as Bcr-Abl inhibitor like imatinib (Gleevec) which is used for treating chronic myeloid leukemia and gastrointestinal stromal tumors, and EGFR inhibitors like Gefitinib (Iressa), Erbtinib (Tarceva), EGFR monoclonal antibody (Erbitux) which are used for treating breast cancers, non-small cell lung cancers and colorectal cancers and other malignant tumors.

A spleen tyrosine kinase (SYK) gene was cloned from pig spleen cDNA in 1991 for the first time, and encoded as a non-receptor protein tyrosine kinase. A human SYK gene is located in a q22 zone of No. 9 chromosome. A SYK protein includes 635 amino acids, playing an important role in autoimmune diseases and hematological malignancies. For instance, the SYK gene may inhibit the proliferation and transfer of breast cancers, melanomas, hepatic cancers and other malignant cells. At present, the SYK inhibitor has been applied in clinical experiments at the phase II/III of rheumatiod arthritis, chronic lymphocytic leukemia and the like. The results thereof are encouraging and the drug safety is good.

In cytology experiments and animal in vivo experiments, multiple SYK small molecule compound inhibitors have been developed, including Entospletinib (GS-9973), Fostamatinib (R788), R406 and PRT062607 (P505-15, BIIB057) HCl. The biological effect trends of these SYK inhibitors are similar, and Entospletinib (GS-9973) has the most stable and significant activity in inhibiting SYK kinase. At present, multiple SYK small molecule compound inhibitors have been applied in phase II/III clinical research on the market; wherein GS-9973 is latest reported to be applied in phase II clinical trials of chronic lymphocytic leukemia, with an effective rate high up to 91%. Moreover, the drugs have a strong SYK targeting specificity, a low off-target rate, and a good biological safety.

Until now, there are as yet no experimental study results indicate the SYK is related to the hepatic fibrosis/hepatic cirrhosis, even nor related experimental data that demonstrates the SYK may be used as a therapeutic target for the hepatic fibrosis/hepatic cirrhosis.

### SUMMARY OF THE INVENTION

The present invention aims to provide an application of SYK serving as a therapeutic target for hepatic fibrosis/hepatic cirrhosis.

Through experiments, the investor has found that a SYK gene has a higher expression during the process of hepatic fibrosis/hepatic cirrhosis, and the progression of hepatic fibrosis is accelerated by promoting activation of the hepatic stellate cells (HSCs); and the progression of hepatic fibrosis/hepatic cirrhosis can be effectively slowed down by using a SYK inhibitor or by interfering with SYK gene expression, both of which have good therapeutic effects.

### BRIEF DESCRIPTION OF THE DRAWINGS

FIG. 1: a SYK-mRNA and protein level expression (A&B) in hepatic cells may be up-regulated after the hepatic cells are infected by hepatitis C viruses (HCVs); compared with a normal hepatic tissue without hepatic fibrosis, the SYK protein expression in a hepatic fibrosis tissue infected by hepatitis B viruses (HBVs) is significantly increased (C);

FIG. 2: in the hepatic stellate cells (HSCs) of LX-2 and TWNT-4, the interference on the SYK gene expression may remarkably reduce HSCs activation indexes (α-SMA and PDGFRβ) and relevant indexes of hepatic fibrosis (A&B); In contrast, overexpression of the SYK gene may promote activation (C) of HSCs;

FIG. 3: after treating two strains of human primary HSCs for 48 hours with the SYK inhibitor GS-9973 (1 μM), influences on mRNA level of activation markers α-SMA and PDGFRβ thereof, and an inhibition effect of cell proliferation (B) at different time points (*, P<0.05; **, P<0.01);

FIG. 4: in a mouse hepatic fibrosis model induced by carbon tetrachloride (CCl₄), a SYK small molecule compound GS-9973 may remarkably inhibit the generation of hepatic collagen (Sirius Red staining), and the expression of activation index α-SMA protein of the HSCs (**, P<0.01; ***, P<0.001); and

FIG. 5: in a rat hepatic fibrosis/hepatic cancer model induced by diethylnitrosamine (DEN), SYK targeted therapy may inhibit the generation of the hepatic collagen and activated state of the HSCs of the rat, and inhibit the formation of a hepatic cancer based upon the hepatic fibrosis (## comparing a neogenic hepatic cancer having a diameter of less than 8 mm, P<0.01)

### DETAILED DESCRIPTION OF THE PREFERRED EMBODIMENT

The technical solution of the present invention will be further described hereinafter with reference to experiments.

Influence of HBV or HCV on SYK Expression in Hepatic Cells or Hepatic Tissues

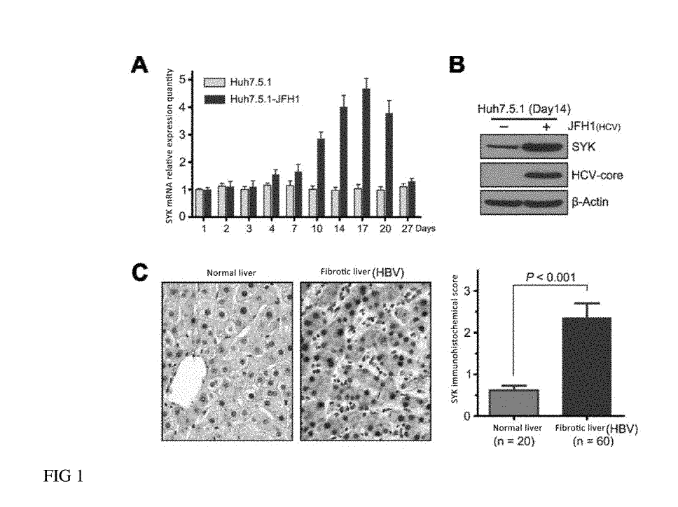

Hepatic cells Huh7.5.1 prone to infect HCV are selected to infect HCV replicon JFH1. A real-time quantitative PCR (qRT-PCR) and Western blot method are employed to detect an influence of HCV on SYK mRNA and protein level in cells respectively. An immunohistochemistry (IHC) method is employed to detect human HBV-related hepatic fibrosis tissues, normal tissues having no hepatic fibrosis and without being infected by HBV/HCV (from a peritumoral hepatic tissue of human's hepatic hemangioma), and compare the SYK protein expression in a hepatic tissue suffered from the hepatic fibrosis and a normal hepatic tissue (FIG. 1).

As can be seen from FIG. 1, a SYK-mRNA and protein level expression (A&B) in hepatic cells may be up-regulated after the hepatic cells are infected by hepatitis C viruses (HCVs); compared with a normal hepatic tissue without hepatic fibrosis, the SYK protein expression in the hepatic fibrosis tissue infected by hepatitis B viruses (HBVs) is significantly increased (C);

Influence of SYK on HSCs Activation and Cell Proliferation

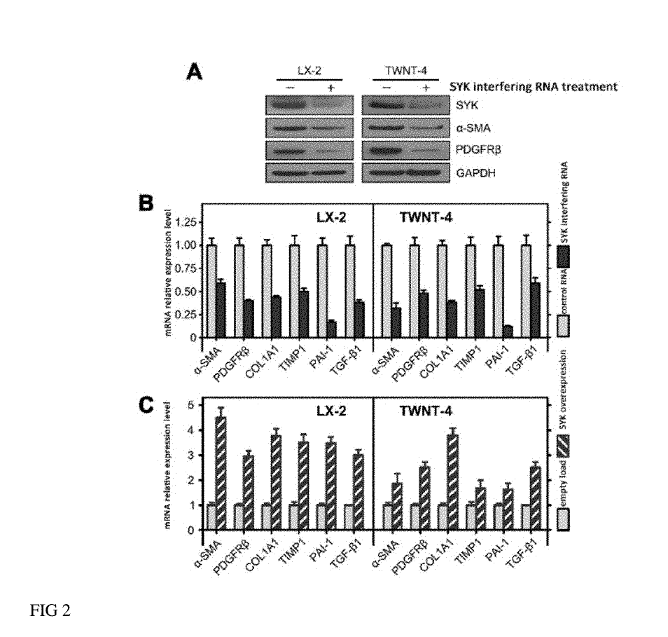

Two strains of human's hepatic stellate cell (HSC) lines of LX-2 and TWNT-4 are selected. SYK gene knockdown or overexpression is employed to treat cells for 48 hours. The qRT-PCR and (or) Western blot method are employed to detect the HSC activation indexes (α-SMA and PDGFRβ), and hepatic fibrosis-related indexes COL1A1, TIMP-1, PAI-1 and TGF-β1 (FIG. 2).

As can be seen from FIG. 2, in the HSCs of LX-2 and TWNT-4, the interference on the gene expression of SYK may remarkably reduce the HSCs activation indexes (α-SMA and PDGFRβ) and relevant indexes of hepatic fibrosis (A&B); In contrast, overexpression of the SYK gene may promote activation (C) of the HSCs.

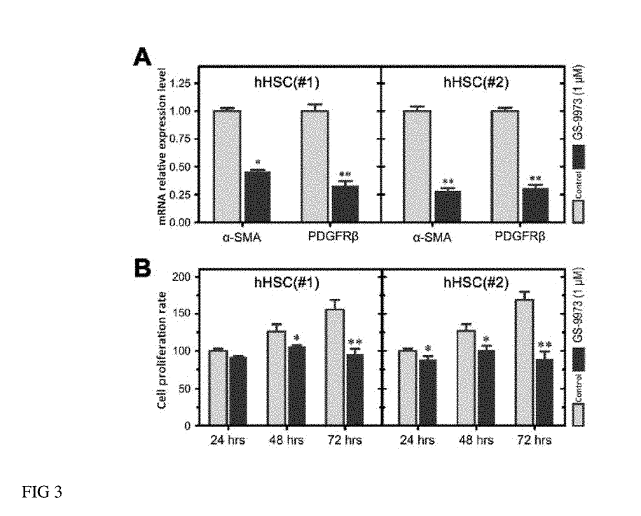

The SYK small molecule compound inhibitor GS-9973 (1 μM) is used for treating two strains of HSCs for 24 hours, 48 hours and 72 hours. CellTiter-Glo Luminescent Cell Viability Assay kit from Promega Corp. is employed to detect the influence of GS-9973 on cell proliferation at different time points. After the inhibitor is used for treating the above-mentioned cells for 48 hours, a qRT-PCR method is employed to detect the mRNA level of HSCs activation indexes (α-SMA and PDGFRβ) (FIG. 3).

As can be seen from FIG. 3, after treating two strains of human primary HSCs for 48 hours, the SYK inhibitor GS-9973 (1 μM) can significantly reduce the mRNA level (A) of the activation markers α-SMA and PDGFRβ thereof and has an obvious inhibition effect on cell proliferation (B) along with the prolongation of the treatment time (*, P<0.05; **, P<0.01).

SYK Targeted Therapy of Hepatic Fibrosis of Animal

Rat hepatic fibrosis/hepatic cancer model: male Wistar rats (body weight: 100-120 g) were used with 8 in each group and were injected intra-abdominally with DEN at 50 mg/kg once a week; and a control group was injected intra-abdominally with an equal volume of PBS for 18 consecutive weeks. The SYK inhibitor GS-9973 is administrated in a manner of gavage at a dose of 2.5 mg/kg once a day starting from the thirteen week and continuously to the seventeen week, with a total of 5 weeks.

Mouse hepatic fibrosis model: male C577BL/6 mice (8-week old) were used with 8 in each group and were injected intra-abdominally with 0.2 ml (diluted by olive oil) of 10% CCl₄ twice a week; and a control group was injected intra-abdominally with an equal volume of olive oil for 18 consecutive weeks. The SYK inhibitor GS-9973 is administrated in a manner of gavage at a dose of 5 mg/kg once a day starting from the thirteen week and continuously the eighteen week, with a total of 6 weeks.

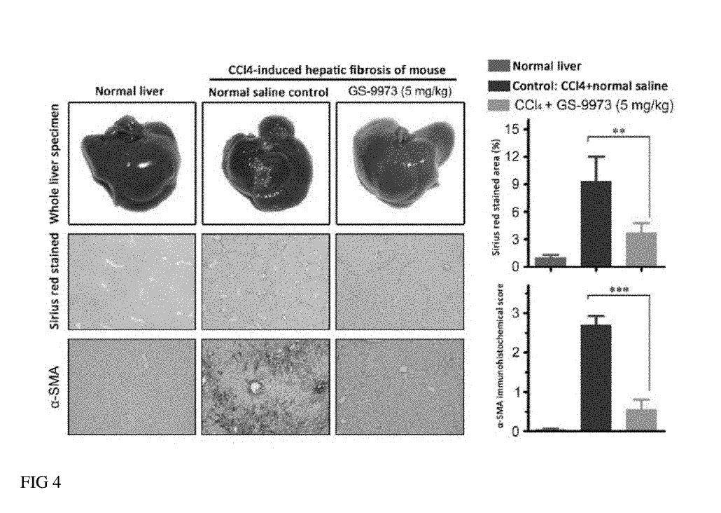

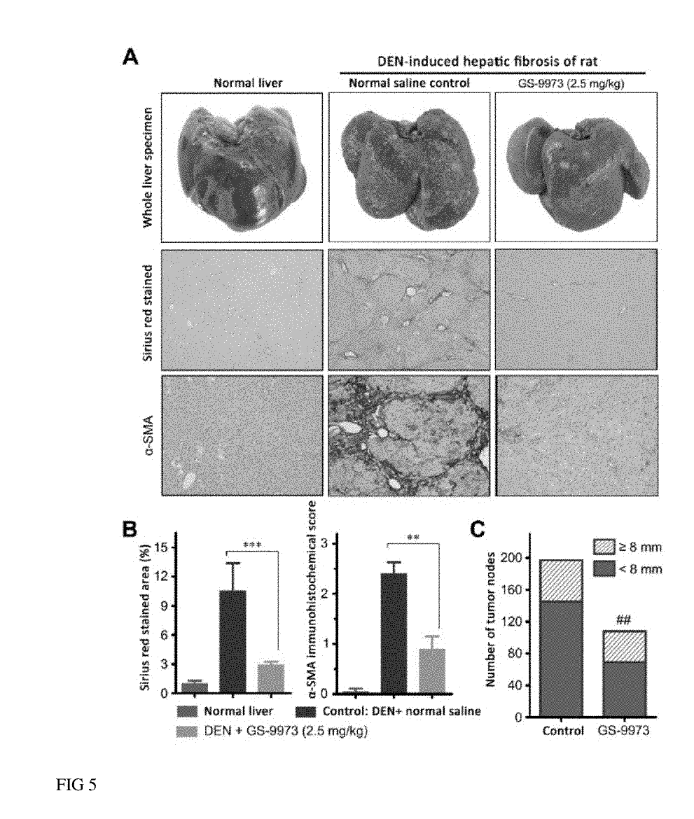

The animals were put to death one week after the last administration of the SYK inhibitor GS-9973. blood was drawn from the inferior vena cava, which was used for serology biochemistry hepatic function test, including ALP, ALT, AST, TBIL and the like; a bit of animal liver was saved as RNA and DNA specimen; a part of medial hepatic tissue was fixed via 4% formalin to prepare a paraffin section; liver tissue histopathological observation was carried out under a light microscope, HE, Sirius Red staining, immunohistochemistry and other methods were used for detecting the hepatic histophathology changes, HSC activation states, hepatic fibrosis indexes and others of the rats (or mice) (FIG. 4 and FIG. 5).

FIG. 4: In a mouse hepatic fibrosis model induced by carbon tetrachloride (CCl₄), SYK small molecule compound GS-9973 may remarkably inhibit the generation of hepatic collagen (Sirius Red staining), and the expression of activation index α-SMA protein of the HSCs (**, P<0.01; ***, P<0.001);

FIG. 5: in a rat hepatic fibrosis/hepatic cancer model induced by diethylnitrosamine (DEN), SYK targeted therapy may inhibit the generation of the hepatic collagen and the activated state of the HSCs of the mice, and inhibit the formation of hepatic cancer based upon the hepatic fibrosis (## comparing a neogenic hepatic cancer having a diameter of less than 8 mm, P<0.01)

It is foreseeable that there is the same therapeutic effect on hepatic fibrosis or hepatic cirrhosis by down-regulating SYK gene expression quantity or inhibiting SYK gene expression, or enabling transcriptional DNA to fail to translate by using nucleic acid interference technology.

Other SYK inhibitors may have therapeutic effects on hepatic fibrosis or hepatic cirrhosis as well.

Therefore, SYK can be served as the therapeutic target for hepatic fibrosis or hepatic cirrhosis.

1. An application of spleen tyrosine kinase (SYK) served as a therapeutic target for hepatic fibrosis/hepatic cirrhosis.

2. The application according to claim 1, characterized in that a treatment of hepatic fibrosis/hepatic cirrhosis is achieved by interfering with SYK gene expression or inhibiting activity of the SYK.

3. An application of compounds or nucleic acids for down-regulating the gene expression of SYK in the manufacture of a medicant for treating or improving hepatic fibrosis/hepatic cirrhosis.

4. The application according to claim 3, characterized in that the nucleic acid for down-regulating the gene expression of SYK is SYK-siRNA, SYK-targeted miRNA or antisense nucleotide.

5. An application of SYK inhibitor in the manufacture of a medicant for treating or improving hepatic fibrosis/hepatic cirrhosis.

6. The application according to claim 5, characterized in that the SYK inhibitor is selected from the group consisting of Entospletinib (GS-9973), Fostamatinib (R788), R406 and PRT062607.

7. A method for treating hepatic fibrosis/hepatic cirrhosis, comprising the administration of a therapeutic amount of at least one of SYK gene expression down-regulation agents and SYK activity inhibitors.

8. The method according to claim 7, characterized in that the SYK gene expression down-regulation agent is selected from the group consisting of SYK-siRNA, SYK-targeted miRNA and antisense nucleotide.

9. The method according to claim 7, characterized in that the SYK activity inhibitor is selected from the group consisting of Entospletinib (GS-9973), Fostamatinib (R788), R406 and PRT062607.
